(19) United States
(12) Patent Application Publication
(10) Pub. No.: US 2025/0387564 A1
(43) Pub. Date: Dec. 25, 2025

(54) An Extended Duration Infusion System

(71) Applicant: University of Galway, Galway (IE)

(72) Inventors: Robert Wylie, Dublin (IE); Jack Connolly, Wicklow (IE); Garry Duffy, Galway (IE); Eimear Dolan, Mayo (IE); Lucien Schreiber, Galway (IE)

(21) Appl. No.: 18/877,735

(22) PCT Filed: Jun. 21, 2023
(86) PCT No.: PCT/EP2023/066863

§ 371 Date: Dec. 20, 2024

(30) Foreign Application Priority Data
Jun. 21, 2022 (EP) 22180265.5

(51) Int. Cl. A61M 5/145; A61M 5/142; A61M 5/172

(52) U.S. Cl. CPC A61M 5/14593; A61M 5/14248; A61M 5/1723; A61M 2202/0468; A61M 2230/201

(57) ABSTRACT

An extended duration infusion set/system suitable for transcutaneous delivery of a treatment fluid to a subject is described. The infusion set reduces and delays fibrotic capsule formation on and around an infusion cannula, and increases the effective lifetime of an infusion set significantly. The infusion set comprises a deflectable therapy reservoir to “actively” diffuse insulin and negate the effects of the foreign body response (FBR).

### FIELD OF THE INVENTION

The present invention relates to an infusion set. The invention also relates to a method of transcutaneously infusing a fluid such as insulin or glucagon into a subject.

### BACKGROUND TO THE INVENTION

Drug infusion sets provide a way of delivering drug transcutaneously to a subject. The infusion sets include a small infusion set cannula attached to a cannula housing which is attached to a patient's body (for example the arm or abdomen), and a pump and associated tubing to deliver drug from a reservoir to the cannula for infusion. The cannula is implanted transcutaneously into the subject. The cannulas are usually part of a wearable device, such as an infusion set or patch pump device and can be used to deliver drug to the subject continuously or at predetermined time intervals. They are commonly used by subjects with diabetes for the delivery of insulin.

A very common problem associated with the current infusion sets is the foreign body response which leads to the formation of a fibrotic layer or capsule on and around the infusion cannula, causing occlusion. This usually occurs within 4 days of implantation, meaning that the cannula has to be removed and discarded, often prematurely, and a new cannula implanted in a different location. Occlusions can also occur due to improper insertion technique, resulting in kinking of the cannula. A further problem with these devices is that pressure can build up in the infusion cannula due to the fibrotic capsule resulting in release of a bolus volume of drug. In the case of insulin infusion sets, such a bolus release can lead to hypoglycaemia in the subject. Failure of insulin delivery could lead to hyperglycaemia and ketoacidosis.

Some companies are attempting to passively negate this response through an increase in diffusion area by increasing the number of pores in the cannula through which insulin can diffuse. This approach can provide some small enhancements in maintaining insulin diffusion over time. However, this incremental approach is passive, and ultimately limited in its effectiveness in overcoming fibrous obstruction and enabling long-term drug diffusion efficacy.

It is an object of the invention to overcome at least one of the above-referenced problems.

### SUMMARY OF THE INVENTION

The problems of the prior art are addressed by providing an infusion set comprising a deflectable therapy reservoir to “actively” diffuse insulin and negate the effects of the foreign body response (FBR). This deflectable therapy reservoir compresses to push the fluid (therapy) within the therapy reservoir down the length of a connected cannula lumen. This compression of the therapy reservoir is controlled by tailoring e.g. the pressure, ramp speed and actuation profile of a second “actuation” reservoir, which uses gas, fluid or a solid body to achieve a plurality of diffusion regimes to deflect the therapy reservoir to overcome the effects of the fibrous capsule during drug diffusion as it develops over time. By negating the effects of this FBR, it is possible to consistently deliver preset doses of drug into the surrounding tissue, change the area of tissue these doses are delivered to, and consistently troubleshoot the positioning of the cannula lumen, for example, straightening the cannula lumen when it becomes kinked. The cannula lumen can have defined pores along its length or can be entirely porous. The provision of a drug reservoir with a fluidic deflector provides a more controllable actuation mechanism allowing more finely tuned and responsive drug delivery from the cannula.

In a first aspect, there is provided an infusion set suitable for transcutaneous delivery of a treatment fluid to a subject, the comprising:
a small infusion set cannula comprising a hollow body defining a central lumen, a fluid inlet, and a plurality of infusion outlets (8) in fluid communication with the central lumen;
a treatment fluid chamber comprising a fluid outlet in fluidic communication with the fluid inlet of the small infusion set cannula and a fluid inlet;
a treatment fluid supply conduit in fluid communication with the fluid inlet of the treatment fluid chamber;
a fluidic deflector comprising an actuation chamber; an actuation fluid storage reservoir, an actuation fluid supply conduit fluidically connecting the actuation fluid storage reservoir to the actuation chamber, and an actuation fluid pump operable to pump actuation fluid from the actuation fluid storage reservoir into the actuation chamber;
a resiliently deformable deflectable membrane separating the treatment fluid in the treatment fluid chamber and the actuation fluid in the actuation chamber; and
a controller operatively connected to the actuation fluid pump and configured to control the actuation of the fluidic deflector by actuation of the actuation fluid pump,

The the fluidic deflector is typically configured to deflect the resiliently deformable deflectable membrane upon actuation to push treatment fluid from the treatment fluid chamber into the central lumen of the small infusion set cannula and out through the infusion outlets.

The infusion set of the invention allows drug to be pumped from a reservoir to an implanted infusion set cannula at a predetermined rate, and also address the problems of fibrous capsule build-up by providing a fluidic deflector to apply an additional “push” to the drug at time intervals and defined pressures which has been found to significantly reduce the rate of fibrotic capsule formation. In addition, the infusion set of the invention allows the fluidic deflector to be positioned adjacent to the cannula, which is advantageous because because it is then in line with the flow of therapy in the infusion system.

In any embodiment, the infusion set comprises a treatment fluid storage reservoir and a treatment fluid pump configured to pump treatment fluid from the treatment fluid storage reservoir to the small infusion set cannula via the treatment fluid supply conduit and the treatment fluid chamber.

In any embodiment, the treatment fluid chamber comprises the resiliently deformable deflectable membrane.

In any embodiment, the treatment fluid chamber is contained within the actuation chamber.

In any embodiment, the treatment fluid chamber and actuation chamber are coupled to a proximal end of the small infusion set cannula.

In any embodiment, the treatment fluid chamber and actuation chamber are configured to be disposed on the skin surface.

In any embodiment, the resiliently deformable deflectable membrane has a convex configuration prior to fluidic deflection.

In any embodiment, the small infusion set cannula has an axial length of 5-20 mm.

In any embodiment, the controller is configured to control a parameter of the actuation of the fluidic deflector selected from the frequency of actuation of the fluidic deflector, the pressure in the treatment fluid chamber during actuation of the fluidic deflector, the ramp rate of the pressure in the treatment fluid chamber during actuation, and the actuation profile/waveform.

In any embodiment, the controller is pre-programmed to actuate the fluidic deflector at timed intervals during the delivery of a dosage of drug.

In any embodiment, the controller is pre-programmed to actuate the fluidic deflector according to a square wave actuation profile, with an on-time of 4-8 second, and an off-time of 4-8 seconds.

In any embodiment, the controller is pre-programmed to actuate the fluidic deflector at an amplitude of 0.5 to 15, 2 to 12, or 4 to 8, psi.

In any embodiment, the infusion set (30) comprises a body parameter sensor.

In any embodiment, the body parameter sensor is operatively connected to the controller, and in which the controller is configured to actuate the fluidic deflector in response to measurements received from the sensor.

In any embodiment, the plurality of infusion outlets are disposed along a length of the small infusion set cannula.

In any embodiment, the small infusion set cannula is flexible.

In any embodiment, the infusion set comprises:
a small infusion set cannula having a hollow body defining a central lumen comprising a plurality of infusion outlets in fluid communication with the central lumen;
a treatment fluid chamber comprising a fluid outlet in fluidic communication with an inlet of the central lumen of the small infusion set cannula;
a treatment fluid storage reservoir;
a treatment fluid supply conduit providing fluidic communication between the treatment fluid storage reservoir and a fluid inlet of the treatment fluid chamber;
a treatment fluid pump configured to pump treatment fluid from the treatment fluid storage reservoir to the treatment fluid chamber through the treatment fluid supply conduit.
a fluidic deflector comprising an actuation chamber, an actuation fluid storage reservoir fluidically connected to the actuation chamber, and an actuation fluid pump operable to pump actuation fluid from the actuation fluid storage reservoir to the actuation chamber; and
a controller operatively connected to the actuation fluid pump and configured to control the actuation of the actuation fluid pump,

Typically, the treatment fluid chamber comprises a resiliently deformable deflectable membrane that separates the treatment fluid in the treatment fluid chamber from the actuation fluid in the actuation fluid chamber.

In any embodiment, the infusion set comprises:
a small infusion set cannula having a hollow body defining a central lumen having a plurality of infusion outlets in fluid communication with the central lumen;
a treatment fluid chamber comprising a fluid outlet in fluidic communication with an inlet of the central lumen of the small infusion set cannula;
a treatment fluid supply conduit in fluid communication with a fluid inlet of the treatment fluid chamber;
a fluidic deflector comprising an actuation chamber, an actuation fluid storage reservoir fluidically connected to the actuation chamber, and an actuation fluid pump operable to pump actuation fluid from the actuation fluid storage reservoir to the actuation chamber; and
a resiliently deformable deflectable membrane separating the treatment fluid in the treatment fluid chamber and the actuation fluid in the actuation chamber; and
a controller operatively connected to the actuation fluid pump and configured to control the fluidic deflector by actuation of the actuation fluid pump,

Typically the controller is pre-programmed to actuate the fluidic deflector at timed intervals during the delivery of a dosage of drug.

In any embodiment, the infusion set comprises:
a small infusion set cannula having a hollow body defining a central lumen having a plurality of infusion outlets in fluid communication with the central lumen;
a treatment fluid chamber comprising a fluid outlet in fluidic communication with an inlet of the central lumen of the small infusion set cannula;
a treatment fluid supply conduit in fluid communication with a fluid inlet of the treatment fluid chamber;
a fluidic deflector comprising an actuation chamber, an actuation fluid storage reservoir fluidically connected to the actuation chamber, and an actuation fluid pump operable to pump actuation fluid from the actuation fluid storage reservoir to the actuation chamber; and
a resiliently deformable deflectable membrane separating the treatment fluid in the treatment fluid chamber and the actuation fluid in the actuation chamber; and
a controller operatively connected to the actuation fluid pump and configured to control the fluidic deflector by actuation of the actuation fluid pump,

Typically, the controller is configured to actuate the fluidic deflector with a square waveform actuation profile.

In another aspect, there is provided an infusion cannula set suitable for transcutaneous delivery of a treatment fluid to a subject, comprising
a small infusion set cannula having a hollow body defining a central lumen having a plurality of infusion outlets in fluid communication with the central lumen;
a treatment fluid chamber defined at least partly by a resiliently deformable deflectable membrane and comprising a fluid outlet in fluid communication with an inlet of the central lumen of the small infusion set cannula;
a treatment fluid supply conduit in fluid communication with a fluid inlet of the treatment fluid chamber;
a fluidic deflector comprising an actuation chamber configured to be pneumatically or hydraulically pressurised by an actuation fluid pump; and
a controller operatively connected to the fluidic deflector and operable to control the actuation of the fluidic deflector,

Typically, the treatment fluid chamber is contained within the actuation chamber such that, during use, pressurisation of the actuation chamber causes the resiliently deformable deflectable membrane to deflect to push treatment fluid from the treatment fluid chamber into the hollow body of the small infusion set cannula and out through the infusion openings.

In another aspect, there is provided an infusion cannula set suitable for transcutaneous delivery of a treatment fluid to a subject, comprising
a small infusion set cannula having a hollow body defining a central lumen comprising a plurality of infusion outlets in fluid communication with the central lumen;
a treatment fluid storage reservoir;
a treatment fluid supply conduit providing fluidic communication between the treatment fluid storage reservoir and a fluid inlet of the small infusion set cannula;
a treatment fluid pump (19) configured to pump treatment fluid from the treatment fluid storage reservoir (18) to the small infusion set cannula (6) via the treatment fluid supply conduit (15);
   characterised in that the infusion set comprises an actuation module comprising:
a fluidic deflector comprising an actuation chamber (11), an actuation fluid storage reservoir (20) fluidically connected to the actuation chamber (11), and an actuation fluid pump (21) operable to pump actuation fluid from the actuation fluid storage reservoir (20) to the actuation chamber (11) to pressurise the actuation chamber (11);
a compressible treatment fluid chamber (10) disposed in-line in the treatment fluid supply conduit (15); and
a controller operatively connected to the actuation fluid pump (21) and configured to control the actuation of the actuation fluid pump (21), wherein the compressible treatment fluid chamber (10) is disposed within the actuation chamber, whereby the actuation chamber (11) when pressurised causes the compressible treatment fluid chamber (10) to compress to push treatment fluid from the treatment fluid chamber (10) into the hollow body of the small infusion set cannula (6) and out through the infusion openings (8).

In another aspect, there is providedan infusion set comprising an infusion set cannula suitable for transcutaneous delivery of a treatment fluid such as a drug to a subject. The term “treatment fluid” should be understood to include fluids intended for therapeutic or diagnostic purposes. The infusion set generally comprises: an infusion set cannula having a hollow body (which is generally flexible) defining a central lumen having a distal end and a proximal end and a plurality of infusion openings in fluid communication with the central lumen;
a treatment fluid reservoir comprising a deflectable membrane and in fluid communication with the central lumen of flexible hollow body;
a treatment fluid supply conduit in fluid communication with the treatment fluid reservoir; and
a deflector configured to be actuated to deflect the deflectable membrane to push treatment fluid from the treatment fluid reservoir into the flexible hollow body of the infusion cannula and out through the infusion openings.

Generally, the treatment fluid reservoir is disposed at a proximal end of the hollow tube. In these embodiments, the treatment fluid reservoir forms part of the implantable part of the cannula. In other embodiments, it is remote to the hollow tube and connected thereto by a fluidic conduit. In these embodiments, the treatment fluid reservoir is generally located outside of the body.

In any embodiment, the treatment fluid reservoir is a resiliently deformable chamber. In any embodiment, the treatment fluid reservoir is a soft robotic capsule. In any embodiment, the deflectable membrane is deformable, typically resiliently deformable.

In any embodiment, the cannula comprises an actuation chamber and the treatment fluid reservoir is disposed within the actuation chamber.

In any embodiment, the deflector is a fluidic deflector.

In any embodiment, the fluidic deflector comprises a deflector chamber, a deflector fluid supply conduit fluidically connected to the deflector chamber, and a first pump operable to pump deflector fluid into the deflector chamber, whereby supply of deflector fluid to the deflector chamber causes the deflector chamber to increase in size and effect deflection of the deflectable membrane.

In any embodiment, the treatment fluid reservoir and deflector chamber are separated by the deflectable membrane.

In any embodiment, the deflector is configured to mechanically deflect the deflectable membrane.

In any embodiment, the deflector is configured to electrically deflect the deflectable membrane.

In any embodiment, the deflector is configured to magnetically deflect the deflectable membrane.

In any embodiment, the deflector is configured to thermally deflect the deflectable membrane (e.g. photothermal actuation or electrothermal actuation).

In any embodiment, the deflectable membrane has a convex configuration.

In any embodiment, the deflectable membrane is deflectable from a convex configuration to a planar or concave configuration.

In any embodiment, the deflectable membrane is deflectable from planar to a concave configuration.

In any embodiment, the treatment fluid supply conduit comprises a lumen disposed within the deflector fluid supply conduit.

In any embodiment, the deflector fluid supply conduit comprises a lumen disposed within the treatment fluid supply conduit.

In any embodiment, the plurality of infusion openings are disposed along a sidewall of the flexible hollow body.

In any embodiment, the plurality of infusion openings are provided by at least a part of the hollow body being formed from a material that is porous to the treatment fluid. Such porous material may be formed by, for example, salt leaching.

In any embodiment, the infusion cannula is dimensioned for transcutaneous implantation to deliver drug sub-dermally or into adipose tissue.

In any embodiment, the infusion cannula has an axial length of 5-20 mm, for example 5-10 mm, 10-20 mm, 10-15 mm or 15-20 mm.

In any embodiment, the infusion cannula has an internal diameter of 0.1 to 1.0 mm or 0.1 to 0.5 mm.

In any embodiment, the system of the invention is wearable.

In any embodiment, the system comprises a treatment fluid storage reservoir fluidically connected to a proximal end of the treatment fluid supply conduit.

In any embodiment, the system comprises a second pump configured to pump treatment fluid from the treatment fluid storage reservoir to the deflectable treatment fluid reservoir upon actuation.

In any embodiment, the system comprises a controller operatively connected to the deflector and operable to control the actuation of the deflector. The controller is configured to control the rate of actuation of the fluidic deflector (e.g. how often it is actuated). The controller may be configured to control the pressure in the treatment fluid reservoir during deflection, the ramp rate of the pressure, and the actuation profile/waveform (see FIG. 7E).

In one embodiment, the controller is pre-programmed to actuate the deflector according to a sequence of steps.

In any embodiment, at least two of the sequential steps comprises a different deflection waveform.

In any embodiment, the controller is configured to perform the sequence of steps a plurality of times per day at predetermined time intervals.

In any embodiment, the system includes a body parameter sensor. The body parameter sensor may be a blood or body fluid sensor. The sensor may be configured to detect a parameter of a body fluid, for example temperature, pH, oxygen, or the level of a component in the body fluid, for example a metabolite such as glucose.

In any embodiment, the sensor is operatively connected to the controller, and in which the controller is configured to actuate the sequence of steps in response to measurements received from the sensor.

In any embodiment, the sensor is a glucose sensor configured to measure glucose in the blood or interstitial fluid of a subject, in which the glucose sensor is operatively connected to the controller, and in which the controller is configured to actuate the sequence of steps in response to blood glucose measurements received from the sensor.

In any embodiment, the sensor is configured for manual actuation of the pumps. This is suitable for diabetes patients who, for example, may wish to actuate the system before a meal to deliver a bolus dose of a fluid such as insulin.

The invention also relates to the use of an infusion set described herein to deliver a fluid to a subject by cannula infusion, typically transcutaneously.

The invention also relates to a method of diffusing a fluid into a subject (that in one embodiment employs an infusion set of the invention), the method comprising the steps of:
implanting an infusion set cannula having infusion outlets in tissue (e.g transcutaneously) of the subject;
actuation of a treatment fluid pump to advance a treatment fluid through a treatment fluid supply conduit and into a treatment fluid chamber; and
actuation of a fluidic deflector by a controller to deflect a resiliently deformable deflectable membrane in fluid communication with treatment fluid in the treatment fluid chamber to push treatment fluid from the treatment fluid chamber into the infusion set cannula and out through the infusion outlets.

In any embodiment, the infusion set cannula is transcutaneously implanted in the subject.

In any embodiment, the fluidic deflector comprises an actuation chamber and a means for fluidically pressurising the actuation chamber with actuation fluid to fluidically deflect the resiliently deformable deflectable membrane, wherein the method comprises actuation of an actuation fluid pump to fluidically pressurise the actuation chamber.

In any embodiment, the method comprises delivering a defined dose of treatment fluid to the subject.

In any embodiment, the resiliently deformable deflectable membrane forms part of the treatment fluid chamber, in which the method comprises implanting the treatment fluid chamber and the actuation chamber sub-dermally.

In any embodiment, the method comprises moditying at least one deflection parameter of the fluidic deflector during the delivery of the defined dose of treatment fluid.

In any embodiment, the deflection parameter is selected from the pressure in the treatment fluid chamber during actuation of the fluidic deflector, the ramp rate of the pressure in the treatment fluid chamber during actuation, and the actuation profile/waveform.

In any embodiment, the method comprises actuation of the fluidic deflector according to a sequence of steps in which at least two of the sequence of steps comprises a different deflection parameter value. For example, if the parameter is the waveform of the fluidic deflector, the first step may be a square actuation waveform and the second step may be a triangular actuation waveform.

In any embodiment, the sequence of steps includes at leasts three steps in which each step comprises a different actuation waveform.

In any embodiment, the method comprises moditying the ramp rate of the pressure in the treatment fluid chamber during actuation during the delivery of the defined dose of treatment fluid,

In any embodiment, the infusion set comprises a controller operatively connected to the actuation fluid pump and configured to control the actuation of the actuation fluid pump during the delivery of the defined dose of treatment fluid.

In any embodiment, the fluid comprises a pharmaceutically active agent.

In any embodiment, the pharmaceutically active agent is insulin or glucagon.

In any embodiment, the inflation fluid is a gas such as air or a liquid such as saline

Other aspects and preferred embodiments of the invention are defined and described in the other claims set out below.

### BRIEF DESCRIPTION OF THE FIGURES

FIG. 1(A): Infusion cannula system of the invention.

FIG. 1(B): Implantable infusion cannula forming part of the infusion cannula system of the invention and comprising hollow tube and actuation chamber

FIG. 2: Infusion cannula with treatment fluid reservoir during treatment fluid filling: (A) prior to filling; (B) during filling; and (C) after filling.

FIG. 3: Infusion cannula with deflection chamber and treatment fluid reservoir during actuation (treatment fluid delivery through cannula): (A) treatment fluid reservoir charged with treatment fluid; (B) deflection chamber is charged with fluid causing the deflectable membrane of the treatment fluid reservoir to deflect pushing treatment fluid into the cannula and out through the holes into the tissue; and (C) after treatment fluid reservoir is re-filled with treatment fluid.

FIG. 4: Examples of different embodiments of the invention including (A) a fluidic deflector for the deflectable membrane (B) electrical, magnetic or sonic deflector for the deflectable membrane (C) mechanical deflector for the deflectable membrane (D) photothermal deflector for the deflectable membrane (E) electrothermal deflector for the deflectable membrane.

FIG. 5: illustration of a system of the invention comprising an infusion cannula system, a glucose monitor and a mobile device configured to communicate with the sensor and a control system and which controls the operation of the cannula based on data received from the glucose sensor.

FIG. 6: (A) Comparison of active vs passive diffusion in terms of drop in Blood glucose level over time. Separate passive (B) and active (C) diffusion blood glucose curves over time.

FIG. 7(A): Diffusion with respect to time of a drug analogue through the fibrous capsule formed around the device 4 days and 20 days after implantation.

FIG. 7(B): Diffusion of a drug analogue (shows in red) through the fibrous capsule formed around a device 21 days after subcutaneous implantation in a rat where the drug analogue is concentrated in the reservoir of the device (broken white lines) before actuation and has diffused into the surrounding tissue after actuation.

FIG. 7(C): Quantification of the diffusion area (cm2) with respect to time for passive diffusion along (blue line) and actuation-assisted diffusion (red line).

FIG. 7(D): Quantification of the diffusion area (cm2) of the drug analogue before actuation, after actuation and for passive diffusion alone, where bars correspond to matching colours in graph in part C.

FIG. 7(E): Modifying the waveform of the actuation regime gives a high level of control of drug delivery profiles.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein, the term “disease” is used to define any abnormal condition that impairs physiological function and is associated with specific symptoms. The term is used broadly to encompass any disorder, illness, abnormality, pathology, sickness, condition or syndrome in which physiological function is impaired irrespective of the nature of the aetiology (or indeed whether the aetiological basis for the disease is established). It therefore encompasses conditions arising from infection, trauma, injury, surgery, radiological ablation, age, poisoning or nutritional deficiencies.

As used herein, the term “treatment” or “treating” refers to an intervention (e.g. the administration of an agent to a subject) which cures, ameliorates or lessens the symptoms of a disease or removes (or lessens the impact of) its cause(s) (for example, the increase in levels of a tight junction protein). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

As used herein, an effective amount or a therapeutically effective amount of an agent defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure. Improvement may be observed in biological/molecular markers, clinical or observational improvements. In a preferred embodiment, the methods of the invention are applicable to humans, large racing animals (horses, camels, dogs), and domestic companion animals (cats and dogs).

In the context of treatment and effective amounts as defined above, the term subject (which is to be read to include “individual”, “animal”, “patient” or “mammal” where context permits) defines any subject, particularly a mammalian subject, for whom treatment is indicated. Mammalian subjects include, but are not limited to, humans, domestic animals, farm animals, zoo animals, sport animals, pet animals such as dogs, cats, guinea pigs, rabbits, rats, mice, horses, camels, bison, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; and rodents such as mice, rats, hamsters and guinea pigs. In preferred embodiments, the subject is a human. As used herein, the term “equine” refers to mammals of the family Equidae, which includes horses, donkeys, asses, kiang and zebra.

“Infusion set cannula” refers to an implantable cannula having a hollow body with infusion outlets configured to allow liquid within the hollow body pass into surrounding tissue in which the cannula is implanted. The infusion cannula is generally a small infusion set cannula (e.g having an axial length of less than 30 or 25 mm and/or an internal diameter of less than 1.5, 1.0, 0.75, 0.5 or 0.3 mm) such as a transcutaneous infusion set cannula which generally has a length of 6-20 mm and an internal diameter of 0.15 to 1.0 mm. In one embodiment, the cannula is a 25G cannula. Infusion set cannula's are more prone to blockage due to fibrotic encapsulation compared with larger cannula's such as central line cannula's. The infusion set cannula is generally flexible. The cannula may be formed of any suitable material, the details of which will be known to a person skilled in the art. Examples of materials include silicone, polyurethane, polyethylene, polyvinylchloride, PTFE and nylon. In one embodiment, the infusion set cannula is not an intravenous cannula. The cannula generally has an open distal end dimensioned to allow a delivery needle be advanced axially through the lumen of the cannula during implantation of the infusion cannula.

“Transcutaneous” as applied to an infusion set cannula means an infusion set cannula configured for administration through the skin and implantation under the skin or in the adipose layer under the skin. Transcutaneous infusion cannulas are often designed for self-implantation by a patient using an applicator (e.g. Medtronic MINIMED™ infusion set), and do not require a physician for implantation.

“Infusion outlets” refer to apertures that allow infusion of fluid from the lumen of the cannula to the surrounding tissue. The apertures generally are small pores formed in the cannula side wall that are generally co-extensive with at least a distal end, and generally the full length, of the balloon. The apertures are generally disposed on a side of the cannula, and optionally also at a distal tip of the cannula. In the embodiments shown, the cannula has two series of four infusion openings disposed axially on opposite sides of the cannula side wall. It will be appreciated that the infusion openings may be provided in any number or arrangement. The openings may have a size ranging from 5 to 300 microns. The pores may be configured so that the cannula has a porosity of 5 to 95% (i.e the % area of the cannula sidewall that is open due to the pores). The infusion openings may also be of any shape, including round holes or elongated slits. The infusion openings may have the same internal diameter from inside to outside, or may be conical having a larger inlet than outlet or vica versa. In some embodiments, the cannula may be formed from a material that is itself porous, where the pores provide the infusion openings. Cannula's that are porous may be formed by porogen leaching techniques (Jeffrey A. Coffer et al PSS Vol 202, Issue 8 Jun. 2005). Porous cannulas may be made from Silicone, Pebax, Polyurethane, Teflon, PTFE, Nylon elastomers, Dacron and other thermoplastic elastomers.

“Treatment fluid chamber” refers to a chamber having a deflectable membrane, an inlet to fluidically connect with a treatment fluid supply conduit, and an outlet to fluidically connect with the lumen of the infusion set cannula. The chamber is generally disposed at a proximal end of the infusion set cannula and is integrally formed with the infusion set cannula and configured for implantation thberewith. In other embodiments, the chamberis remote to the infusion set cannula and connected thereto by a length of tubing. The chamber generally takes the form of a soft robotic capsule comprising a resiliently deformable deflectable membranne. The deflectable membrane may be convex and configured for deflection by the fluidic deflector into a planar or concave configuration.

“Fluidic deflector” refers to an apparatus configured to deflect the deflectable membrane of the treatment fluid reservoir upon actuation. Generally, the fluidic deflector comprises an actuation chamber that is fluidically connected to an actuation fluid supply conduit. Pumping actuation fluid, generally a liquid such as saline, into the chamber causes the deflectable membrane of the treatment fluid reservoir to deflect and push treatment fluid into the hollow tube of the infusion cannula. The deflectable membrane may be convex, for example dome shaped. The deflectable membrane may be deflectable from a convex shape to a planar or concave shape (or a less convex shape). The actuation chamber may include an actuation fluid reservoir that is fluidically connected to the actuation fluid supply conduit and is configured to bear against the deflectable membrane when actuation fluid is pumped into the actuation fluid reservoir. In other embodiments, the deflector may an electrical or electro-mechanical apparatus configured to deflect the deflectable membrane when actuated. In other embodiments, the deflector may form part of the deflectable membrane and be actuatable by electrical, magnetic, sonic, or thermal (photothermal or electrothermal) means to cause the membrane to deflect. In one embodiment, the deflectable membrane comprises a dielectric elastomer or a piezoelectric material.

“Fluid” refers to an air, gas or liquid or mixture thereof. When the deflector is fluidic, the fluid is generally a liquid such as saline, but may also be air. The treatment fluid is generally a drug, for example a drug that is self-administered such as insulin, or a diagnostic reagent. Other types of drug that may be self-administered are anti-inflammatory, anti-microbial, pain medicaments, and chemotherapeutic drugs. The drug is generally fluid and may be a solution or suspension.

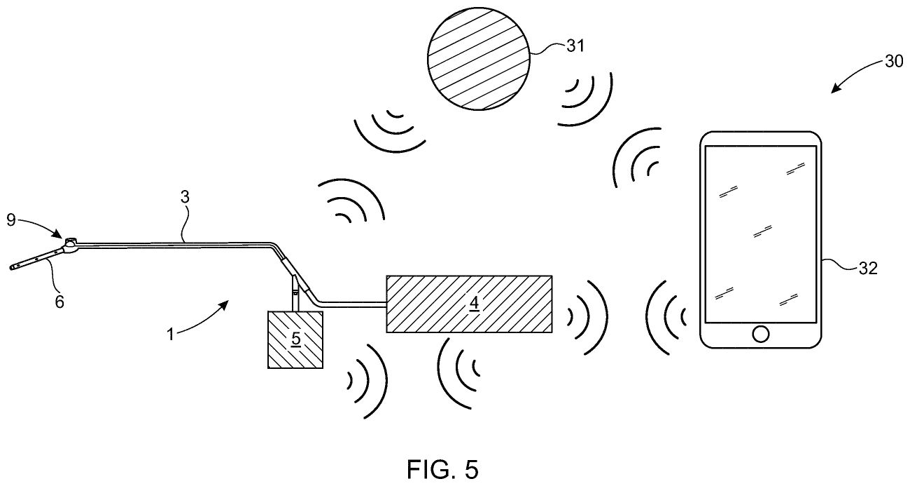

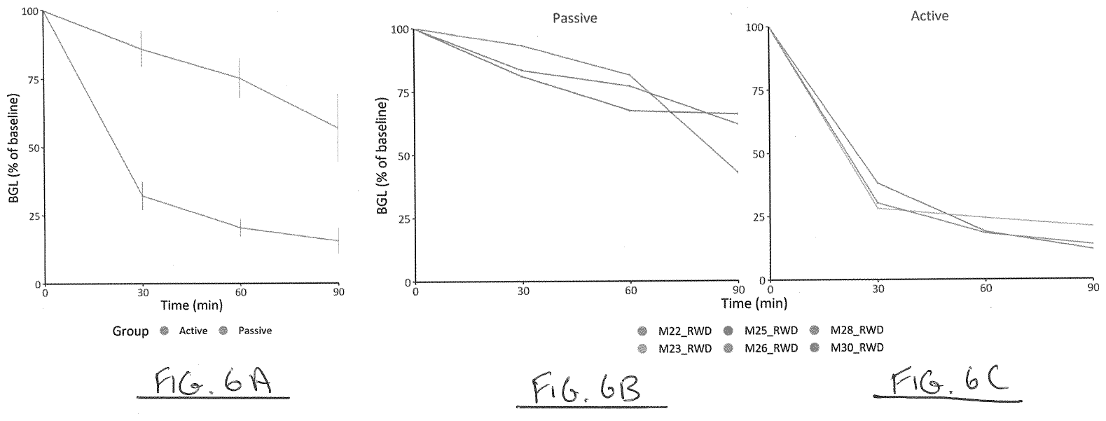

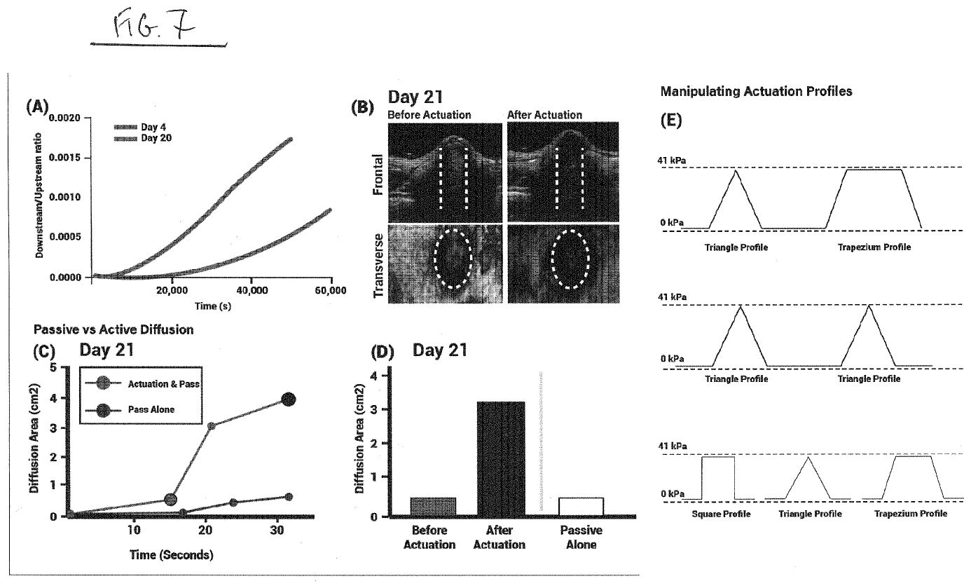

“Controller” refers to a device operatively connected to the actuation fluid pump and configured to control the actuation of the pump. The system generally includes a pump for the actuation fluid supply conduit (actuation fluid pump) and a separate pump for the treatment fluid supply conduit (treatment fluid pump). The Controller may be configured to actuate the pump to re-fill the treatment fluid reservoir after it has been emptied. The Controller may be configured to actuate the actuation fluid pump to push treatment fluid into the infusion set catheter. The controller may be configured to control one or more of the following actuation parameters: time of actuation; frequency of actuation; ramp speed of actuation; max pressure (Pmax) of actuation; time at pmax. In one embodiment the controller is configured to actuate the actuation fluid pump to perform a sequence of actuation steps. Typically at least two (and preferably all) of the actuation steps have a different actuation profile. The actuation profile of the actuation steps may differ in the actuation waveform (See FIG. 5E), which may be selected from square, triangular, trapezium. The controller may be programmable to actuate the system at predetermined times, for example once a day, several times a day, or as required by the user. In addition, the parameters of actuation may be controlled by the controller, for example the volume of drug to be delivered, the drug infusion time, and pressure of drug delivery, and the time period between balloon inflation and balloon deflation. The controller generally comprises a processor operably connected to the controller. The processor may be pre-programmable. The controller may include a graphic display. The controller may include a user interface. The controller and processor may be contained within the same housing. The controller is generally wearable. The controller may include a wireless communications module configured to communicate with a separate processor. The separate processor may be a mobile communications device comprising software comprising instructions for the processor to control the operation of the controller. The software may be downloadable software (e.g. a mobile communications “app”). The software may be configured to graphically display data relating to the operation of the system on a screen of the controller or a mobile communications device.

“Sensor” refers to a device that can measure a body parameter, for example, pH, or the presence or abundance of an element (such as a drug or metabolite) in the body and especially in a body fluid such as blood or interstitial fluid. The sensor may be implantable. Examples include sensors for detecting the level of a metabolite in a body fluid such as blood or interstitial fluid, for example a glucose sensor. The system of the invention may include a sensor, or be configured for operative connection with a sensor. The controller forming part of the system of the invention may be operatively connected to the sensor and actuate the system in response to data received from the sensor. The controller may be configured to actuate the system of the invention when the level of a metabolite reaches a threshold level. For example, when the system of the invention is for infusing insulin, the system may be configured to actuate in response to data from the sensor indicating that the glucose levels have reached a defined threshold level.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

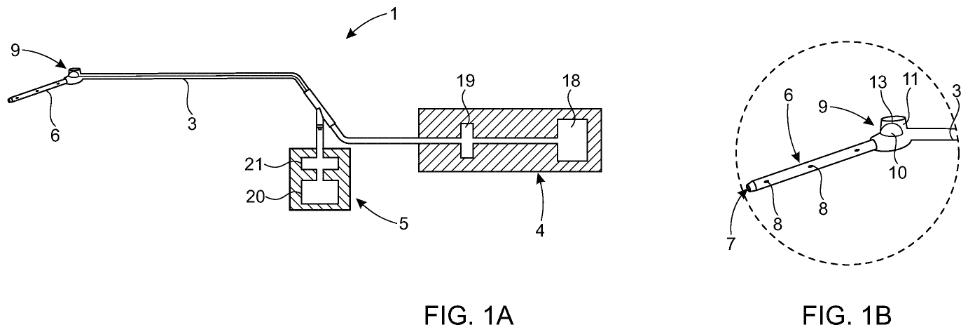

Referring to the drawings and initially to FIGS. 1 to 3, there is illustrated an infusion set of the invention for use in transcutaneously infusing insulin to a subject and indicated generally by the reference numeral 1. The infusion set 1 comprises anl infusion set cannula 6, actuation module 9, drug supply module 4, actuation fluid supply module 5 and fluid supply conduit 3.

The infusion set cannula 6 comprises a flexible tube with an internal lumen 7 and outlet apertures 8. In this embodiment, the infusion set cannula 1 has a length of about 3 cm and a width of about 0.3 cm. Thus, it would be described as a small infusion set cannula. It is dimensioned to be implanted under the skin of a patient and to deliver drug subcutaneously upon actuation.

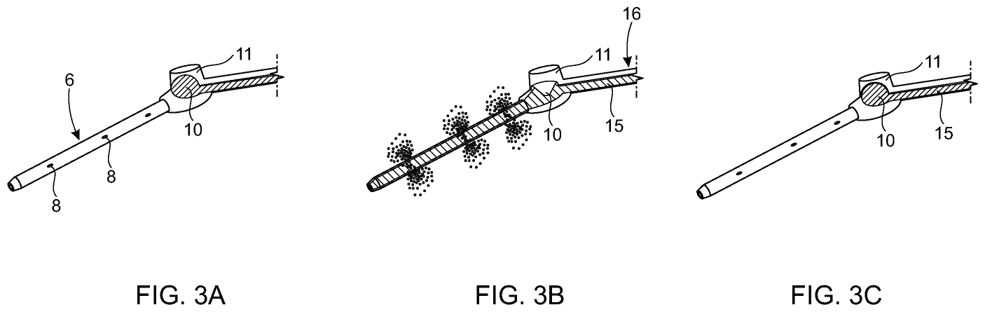

The actuation module 9 is connected to a proximal end of the cannula 6 and comprises an actuation chamber 11 and a drug chamber 10. The actuation chamber 11 is fluidically connected to the actuation fluid supply module 5 (as described below). The drug chamber 10 is fluidically connected at one end to the drug supply module 4 (as described below) and fluidically connected at an opposed end to the internal lumen 7 of the cannula 6. The drug chamber 10 is disposed in contact with the actuation chamber 11 and comprises a dome-shaped (convex) resiliently deformable deflectable membrane 13 that separates the drug in the drug chamber 10 from actuation fluid in the actuation chamber 11. As illustrated in FIGS. 3A and 3B, when actuation fluid is advanced into the actuation chamber 11 to pressurise the chamber, the resiliently deformable membrane 13 is deflected by the pressure a convex shape (FIG. 3A) to a concave shape (FIG. 3B). This has the effect of pushing drug from the drug chamber into the cannula 6 as indicated in FIG. 3B.

Referring specifically to FIG. 1A, the fluid supply conduit 3 connects the infusion set cannula 6 (which in use is located under the skin) with the other parts of the system (which are disposed outside the body). The conduit 3 comprises a drug supply conduit 15 that fluidically connects the drug chamber 10 with the drug supply module 4 and an actuation fluid supply conduit 16 that fluidically connects the actuation chamber 11 with the actuation fluid supply module 5. The drug supply module 4 comprises a drug storage reservoir 18 and a drug pump 19 configured to pump the drug from the storage chamber 18 to the cannula 6 via the drug reservoir 10 upon actuation. The actuation fluid supply module 5 comprises an actuation fluid storage reservoir 20 and an actuation fluid pump 21 configured to pump actuation fluid from the storage reservoir 20 to the actuation chamber 11 upon actuation.

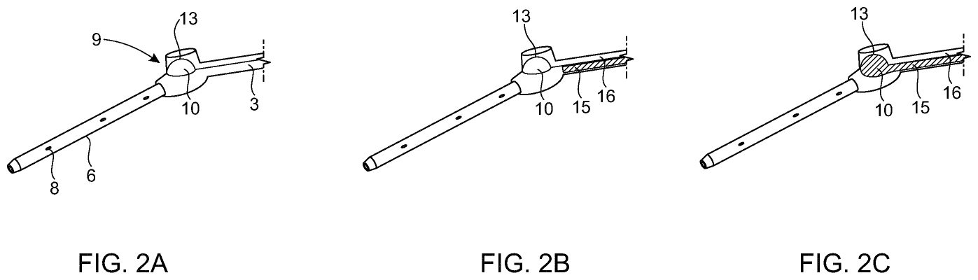

In use, and referring to FIGS. 2 and 3, when the drug pump 19 is actuated drug is pumped from the storage chamber 18 to the drug reservoir 10 to charge the reservoir with drug as shown in FIG. 2C. Although not shown, the drug will continue to the cannula 6 and out of the outlets 8. The pump 19 is configured to deliver drug to the cannula 6 at a set flow rate and until a predetermined dose has been delivered. The actuation fluid pump 21 is then actuated to pump actuation fluid into the actuation chamber 11, which pressurises the chamber and causes deflection of the deflectable membrane 13 from the dome shape shown in FIG. 3A to the concave shape of FIG. 3B, thereby pushing drug into the cannula 6 and out through the infusion outlets 8. During the delivery of the predetermined dose of the drug, the pump is actuated at intervals to intermittently pressurise the actuation chamber at a defined frequency, amplitude (chamber pressure) and waveform. For example, in one embodiment, the actuation chamber is pressurised according to a square waveform (6 seconds on, six seconds off) with an amplitude of 6 psi (e.g. pressure in actuation chamber). It will be appreciated that the profile of pressurisation of the actuation chamber may employ a different amplitude and a different waveform and frequency, depending on the the level of occlusion of the cannula and other parameters, for example the rate of infusion of drug or the size of the infusion area. The profile of the profile of pressurisation of the actuation chamber may be controlled by a controller that is operatively coupled to the actuation fluid pump 21 and configured to actuate the pump by selecting one or more pre-set frequencies of actuation, actuation waveforms, and actuation amplitudes.

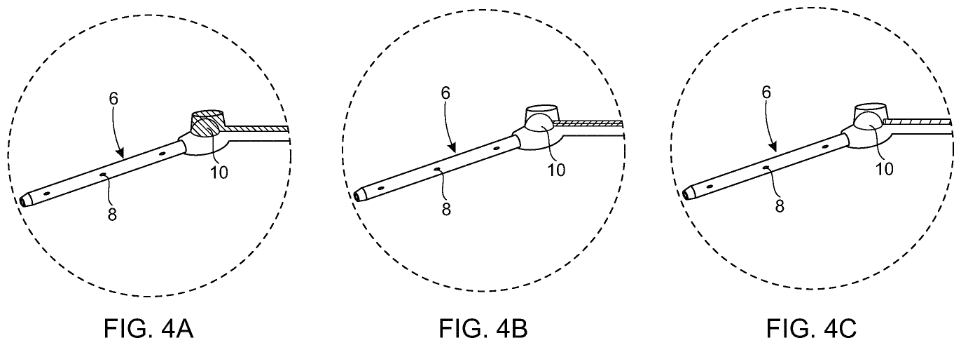

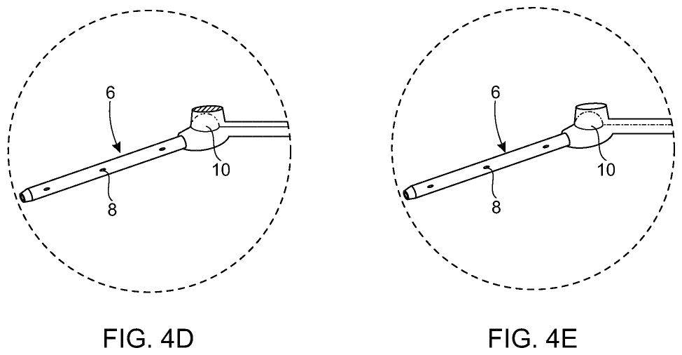

FIG. 4 illustrates different embodiments of the infusion set of the invention including (A) a fluidic deflector for the deflectable membrane (B) electrical, magnetic or sonic deflector for the deflectable membrane (C) mechanical deflector for the deflectable membrane (D) photothermal deflector for the deflectable membrane (E) electrothermal deflector for the deflectable membrane.

FIG. 5 illustrates an infusion set of the invention indicated generally by the reference numeral 30 and in which parts identified with reference to the previous embodiments are assigned the same reference numerals. The system comprises the infusion set 1 described above, a wearable glucose sensor 31 configured to measure blood glucose levels of a subject, and a controller 32 configured to receive data from the sensor wirelessly and then actuate the infusion set 1 based on the received data. In this embodiment, the controller 32 is provided by a mobile phone with controlling software running on the device. The software may be downloadable software (e.g. an “app”). The software may comprise program instructions to instruct the device to communicate wirelessly with the sensor and the two pumps 19, 21 of the infusion set 11, to actuate the actuation pump according to a pre-programmed sequence of steps. The sequence chosen by the controller may be determined by data received from the sensor, or according o the drug being delivered. The controller may control the actuation of the infusion cannula, for example when it actuates (e.g. at what time of the day, how many times per day) and how it actuates (e.g. actuation profile). The controller may be wearable. The system of the invention may be wearable. For example, the controller may be configured to actuate the infusion cannula in a sequence of actuation steps, for example 2 to 100 sequential actuation steps. The sequence of steps may be performed over a period of time, for example, 1 to 60 minutes. At least two of the actuation steps (or most or all) may have a different actuation profile, e.g. square, triangular, trapezium shaped.

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. An infusion set (1) suitable for transcutaneous delivery of a treatment fluid to a subject, the comprising:
a small infusion set cannula (6) comprising a hollow body defining a central lumen (7), a fluid inlet, and a plurality of infusion outlets (8) in fluid communication with the central lumen;
a treatment fluid chamber (10) comprising a fluid outlet in fluidic communication with the fluid inlet of the small infusion set cannula (6) and a fluid inlet;
a treatment fluid supply conduit (15) in fluid communication with the fluid inlet of the treatment fluid chamber (10);
a fluidic deflector comprising an actuation chamber (11); an actuation fluid storage reservoir (20), an actuation fluid supply conduit (16) fluidically connecting the actuation fluid storage reservoir (20) to the actuation chamber (11), and an actuation fluid pump (21) operable to pump actuation fluid from the actuation fluid storage reservoir (20) into the actuation chamber (11);
a resiliently deformable deflectable membrane (13) separating the treatment fluid in the treatment fluid chamber (10) and the actuation fluid in the actuation chamber (11); and
a controller operatively connected to the actuation fluid pump (21) and configured to control the actuation of the fluidic deflector by actuation of the actuation fluid pump (21),
wherein the fluidic deflector is configured to deflect the resiliently deformable deflectable membrane (13) upon actuation to push treatment fluid from the treatment fluid chamber (10) into the central lumen (7) of the small infusion set cannula (6) and out through the infusion outlets (8).

2. An infusion set according to claim 1, including a treatment fluid storage reservoir (18) and a treatment fluid pump (19) configured to pump treatment fluid from the treatment fluid storage reservoir (18) to the small infusion set cannula (6) via the treatment fluid supply conduit (15) and the treatment fluid chamber (10).

3. An infusion set according to claim 1, in which the treatment fluid chamber (10) comprises the resiliently deformable deflectable membrane (13).

4. An infusion set according to claim 1, in which the treatment fluid chamber (10) is contained within the actuation chamber (11).

5. An infusion set according to claim 1, in which the treatment fluid chamber (10) and actuation chamber (11) are connected to a proximal end of the small infusion set cannula (6).

6. An infusion set according to claim 1, in which the treatment fluid chamber (10) and actuation chamber (11) are connected to a proximal end of the small infusion set cannula (6), in which the treatment fluid chamber (10) and actuation chamber (11) are configured to be disposed on the skin surface when the small infusion set cannula (6) is implanted under the skin.

7. An infusion set according to claim 1, in which the resiliently deformable deflectable membrane (13) has a convex configuration prior to fluidic deflection.

8. An infusion set according to claim 1, in which the small infusion set cannula (6) has an axial length of 5-20 mm.

9. An infusion set according to any preceding claim 1, in which the controller is configured to control a parameter of the actuation of the fluidic deflector selected from the frequency of actuation of the fluidic deflector, the pressure in the treatment fluid chamber during actuation of the fluidic deflector, the ramp rate of the pressure in the treatment fluid chamber during actuation, and the actuation profile/waveform.

10. An infusion set according to claim 1, in which the controller is pre-programmed to actuate the fluidic deflector at timed intervals during the delivery of a dosage of drug.

11. An infusion set according to claim 1, in which the controller is pre-programmed to actuate the fluidic deflector according to a square wave actuation profile, with an on-time of 4-8 second, and an off-time of 4-8 seconds.

12. An infusion set according to claim 1, in which the controller is pre-programmed to actuate the fluidic deflector at an amplitude of 4 to 8 psi.

13. An infusion set (30) according to claim 1, comprising a body parameter sensor (31).

14. An infusion set according to claim 1, comprising a body parameter sensor (31) operatively connected to the controller (32), and in which the controller is configured to actuate the fluidic deflector in response to measurements received from the sensor.

15. An infusion set according to claim 1, in which the plurality of infusion outlets (8) are disposed along a length of the small infusion set cannula (6).

16. An infusion set according to any preceding claim 1, in which the small infusion set cannula (6) is connected to the treatment fluid chamber (10) and actuation chamber (11) by means of a flexible or rigid fluidic conduit.

17. An infusion set (1) according to claim 1, comprising:
a small infusion set cannula (6) having a hollow body defining a central lumen (7) comprising a plurality of infusion outlets (8) in fluid communication with the central lumen;
a treatment fluid chamber (10) comprising a fluid outlet in fluidic communication with an inlet of the central lumen of the small infusion set cannula (6);
a treatment fluid storage reservoir (18);
a treatment fluid supply conduit (15) providing fluidic communication between the treatment fluid storage reservoir (16) and a fluid inlet of the treatment fluid chamber (10);
a treatment fluid pump (19) configured to pump treatment fluid from the treatment fluid storage reservoir (18) to the treatment fluid chamber (10) through the treatment fluid supply conduit.
a fluidic deflector comprising an actuation chamber (11), an actuation fluid storage reservoir (20) fluidically connected to the actuation chamber (11), and an actuation fluid pump (21) operable to pump actuation fluid from the actuation fluid storage reservoir (20) to the actuation chamber (11); and
a controller operatively connected to the actuation fluid pump (21) and configured to control the actuation of the actuation fluid pump (21),
wherein:
the treatment fluid chamber (10) comprises a resiliently deformable deflectable membrane (13) that separates the treatment fluid in the treatment fluid chamber from the actuation fluid in the actuation fluid chamber (11).

18. An infusion set according to claim 1, comprising:
a small infusion set cannula (6) having a hollow body defining a central lumen (7) having a plurality of infusion outlets (8) in fluid communication with the central lumen;
a treatment fluid chamber (10) comprising a fluid outlet in fluidic communication with an inlet of the central lumen (7) of the small infusion set cannula (6);
a treatment fluid supply conduit (15) in fluid communication with a fluid inlet of the treatment fluid chamber (10);
a fluidic deflector comprising an actuation chamber (11), an actuation fluid storage reservoir (20) fluidically connected to the actuation chamber (11), and an actuation fluid pump (21) operable to pump actuation fluid from the actuation fluid storage reservoir (20) to the actuation chamber (11); and
a resiliently deformable deflectable membrane (13) separating the treatment fluid in the treatment fluid chamber (10) and the actuation fluid in the actuation chamber (11); and
a controller operatively connected to the actuation fluid pump (21) and configured to control the fluidic deflector by actuation of the actuation fluid pump (21),
in which the controller is pre-programmed to actuate the fluidic deflector at timed intervals during the delivery of a dosage of drug.

19. An infusion set according to claim 1, comprising:
a small infusion set cannula (6) having a hollow body defining a central lumen (7) having a plurality of infusion outlets (8) in fluid communication with the central lumen;
a treatment fluid chamber (10) comprising a fluid outlet in fluidic communication with an inlet of the central lumen (7) of the small infusion set cannula (6);
a treatment fluid supply conduit (15) in fluid communication with a fluid inlet of the treatment fluid chamber (10);
a fluidic deflector comprising an actuation chamber (11), an actuation fluid storage reservoir (20) fluidically connected to the actuation chamber (11), and an actuation fluid pump (21) operable to pump actuation fluid from the actuation fluid storage reservoir (20) to the actuation chamber (11); and
a resiliently deformable deflectable membrane (13) separating the treatment fluid in the treatment fluid chamber (10) and the actuation fluid in the actuation chamber (11); and
a controller operatively connected to the actuation fluid pump (21) and configured to control the fluidic deflector by actuation of the actuation fluid pump (21),
in which the controller is configured to actuate the fluidic deflector with a square waveform actuation profile.

20. An infusion cannula set (1) suitable for transcutaneous delivery of a treatment fluid to a subject, comprising
a small infusion set cannula (6) having a hollow body defining a central lumen having a plurality of infusion outlets (8) in fluid communication with the central lumen;
a treatment fluid chamber (10) defined at least partly by a resiliently deformable deflectable membrane (13) and comprising a fluid outlet in fluid communication with an inlet of the central lumen of the small infusion set cannula (6);
a treatment fluid supply conduit (15) in fluid communication with a fluid inlet of the treatment fluid chamber (10);
a fluidic deflector comprising an actuation chamber (11) configured to be pneumatically or hydraulically pressurised by an actuation fluid pump (21); and
a controller operatively connected to the fluidic deflector and operable to control the actuation of the fluidic deflector,
in which the treatment fluid chamber (10) is contained within the actuation chamber (11) such that, during use, pressurisation of the actuation chamber (11) causes the resiliently deformable deflectable membrane (13) to deflect to push treatment fluid from the treatment fluid chamber (10) into the hollow body of the small infusion set cannula (6) and out through the infusion openings (8).

21. An infusion set (1) according to claim 1, comprising:
a small infusion set cannula (6) having a hollow body defining a central lumen (7) comprising a plurality of infusion outlets (8) in fluid communication with the central lumen;
a treatment fluid storage reservoir (18);
a treatment fluid supply conduit (15) providing fluidic communication between the treatment fluid storage reservoir (16) and a fluid inlet of the small infusion set cannula (6), the treatment fluid supply conduit (15);
a treatment fluid pump (19) configured to pump treatment fluid from the treatment fluid storage reservoir (18) to the small infusion set cannula (6) via the treatment fluid supply conduit (15);
characterised in that the infusion set comprises an actuation module comprising:
a fluidic deflector comprising an actuation chamber (11), an actuation fluid storage reservoir (20) fluidically connected to the actuation chamber (11), and an actuation fluid pump (21) operable to pump actuation fluid from the actuation fluid storage reservoir (20) to the actuation chamber (11) to pressurise the actuation chamber (11);
a compressible treatment fluid chamber (10) disposed in the treatment fluid supply conduit (15); and
a controller operatively connected to the actuation fluid pump (21) and configured to control the actuation of the actuation fluid pump (21),
wherein the compressible treatment fluid chamber (10) is disposed within the actuation chamber, whereby the actuation chamber (11) when pressurised causes the compressible treatment fluid chamber (10) to compress to push treatment fluid from the treatment fluid chamber (10) into the hollow body of the small infusion set cannula (6) and out through the infusion openings (8).
